(19) United States
(12) Patent Application Publication
(10) Pub. No.: US 2026/0034157 A1
(43) Pub. Date: Feb. 5, 2026

(54) TOOLS TO TARGET NATURAL AND SYNTHETIC NUCLEOTIDE-SENSING PATHWAYS

(71) Applicant: THE REGENTS OF THE UNIVERSITY OF CALIFORNIA, Oakland, CA (US)

(72) Inventors: Jeffery S. COX, San Francisco, CA (US); Jay D. KEASLING, Berkeley, CA (US); Michael J. CRONCE, Berkeley, CA (US)

(21) Appl. No.: 19/355,170

(22) Filed: Oct. 10, 2025

Related U.S. Application Data
(63) Continuation-in-part of application No. PCT/US2024/023941, filed on Apr. 10, 2024.
(60) Provisional application No. 63/495,532, filed on Apr. 11, 2023.

(51) Int. Cl. A61K 31/7068; A61K 31/7076; A61P 31/00; A61P 35/00

(52) U.S. Cl. CPC A61K 31/7068; A61K 31/7076; A61P 31/00; A61P 35/00

(57) ABSTRACT

The present invention provides for compositions and methods for deploying ddhNTPs as immunomodulatory therapeutics to modulate P2 receptors, as nucleotidase inhibitors, for applications like host-acting anti-infectives, oncolytics, anti-aging agents, or tissue regeneration agents.

### CROSS-REFERENCE TO RELATED APPLICATIONS

This application claims priority as a continuation-in-part application of PCT International Patent Application No. PCT/US2024/023941, filed Apr. 10, 2024, which in turn claims priority to U.S. Provisional Patent Application Ser. No. 63/495,532, filed Apr. 11, 2023, which are hereby incorporated by reference.

### STATEMENT OF GOVERNMENTAL SUPPORT

The invention was made with government support under Contract No. DE-AC02-05CH11231 awarded by the U.S. Department of Energy, and Grant Nos. AI063302, AI120694, and AI124619 from the National Institutes of Health. The government has certain rights in the invention.

### REFERENCES TO SUBMISSION OF A SEQUENCE LISTING

Reserved.

### FIELD OF THE INVENTION

The present invention is in the field of synthetic nucleotides.

### BACKGROUND OF THE INVENTION

The host-pathogen arms race laid the foundation for many central mechanisms that drive innate immunity and host protection from injury. Among the most ancient contributors to host immunity is Viperin, an Interferon Stimulated Gene (ISG) reported to offer protection against a wide range of eukaryotic viral pathogens, including CHIKV, HCMV, HCV, DENV, WNV, SINV, influenza, and HIV.

While the functional importance of Viperin was widely characterized through transgenic models, the biochemical mechanisms driving broad-spectrum host defense remained elusive for decades (refs). Then, two seminal papers offered glimpses into the mechanisms underpinning Viperin's function: the first study by Fenwick et al. (2017) discovered Viperin was a Radical SAM enzyme that likely accepted a nucleotide as a co-substrate. In the following year, Gizzi et al. (2018) found Viperin catalyzed a reaction converting the ribonucleotide cytidine triphosphate (CTP) into 3′-deoxy-3′,4′-didehydro-CTP (ddhCTP). This work went on to demonstrate exogenous addition of ddhC was sufficient to inhibit Zika virus infection in vitro and offered evidence indicating ddhCTP acted as a chain terminator that disrupted flavivirus RNA-dependent RNA polymerase (RdRP) function.

Early studies arguing ddhCTP to be pathogen-targeting conflict with other reports demonstrating Viperin inhibits both bacterial pathogens and DNA viruses, like HCMV and KSHV (ref). One recent report demonstrated Viperin exhibits antibacterial activity against S. flexneri and L. monocytogenes, and this protection was dependent on the enzyme's catalytic activity (Helbig et al., 2019). Conversely, another study demonstrated that Viperin acts to promote M. tuberculosis infection severity by suppressing NFkB activity (Zhou et al, 2023). Yet another study attributed Viperin's Radical SAM enzyme activity to be independent of ddhCTP (Bai et al., 2019). Thus, numerous studies published since ddhCTP was initially described suggest our mechanistic understanding of Viperin-mediated host protection is incomplete.

Viperin is also peculiar in that homologs can be found across all domains of life, providing evidence the Viperin protein family is of broad importance to unicellular and multicellular organism survival. Viperin homologs can also be found in microbes that inhabit organism-resident and environmental microbiomes, suggesting organisms are frequently interacting with the ddhNTP chemical family. Reports describing the phylogenetic relationship of Viperin-like enzymes imply higher eukaryotes co-evolved with this enzyme in order to mediate host response to infection and injury. To date, it remains unclear whether higher eukaryotes have evolved secondary mechanisms designed to sense ddhCTP, and if so, what physiological consequence these molecules exact on cell physiology.

Relevant literature includes WO2020202142.

### SUMMARY OF THE INVENTION

We disclose that ddhCTP elicits broad-spectrum antimicrobial activity by agonizing nucleotide-activated P2 receptors and that the ddhA nucleoside exhibits more robust protection than either ddhC or ddhG. This invention provides rational design and synthetic and semisynthetic bioproduction of non-natural ddhCTP/ddhATP derivatives that more robustly activate nucleotide receptors and modulate physiology. Synthetic methods are based on Lee, et al., ACS Bio Med Chem Au. 2023 Aug. 16; 3 (4): 322-326, Chemoenzymatic Synthesis of 3′-Deoxy-3′,4′-didehydro-cytidine triphosphate (ddhCTP). Synthetic and semisynthetic methods are further described herein. These sustainable platforms for producing non-naturally occurring nucleotide derivatives generate novel compounds that improve host defense and promote tissue regeneration.

The synthetic Viperins (SynVips) described herein can also be used for in situ production of ddhNTP derivatives as a next-generation approach for cell therapy. Additionally, the invention provides design synthetic, bio-orthogonal control systems for living cell therapies for human health.

Prior indications of ddhNTPs were premised on these molecules acting by directly inhibiting viral replication via interfering with viral replication machinery (RNA-dependent RNA polymerases). This “chain terminator” activity is thought to be reliant on the lack of a 3′ hydroxyl functional group. We found that ddhCTP actually functions by activating P2 receptors, and thus if there are no nucleotide receptors present there is no therapeutic activity. For example, our results shows that ddhCTP is a potent ligand for specific nucleotide receptors (P2RY1, P2RY2, P2RY4, and P2RX4), and these findings are corroborated by the infection data.

We also found that ddhCTP is preferentially targeted by ENTPD8. We disclose this structure-activity relationship for both classes of therapeutic targets (P2 Receptors and nucleotidases) is dependent on the unique stereochemistry of the molecule (specifically the rigid sugar pucker on the ribose ring). These targets provide new and unexpected applications of ddhNTPs including as therapeutics, genetic control systems for eukaryotic systems (living cell therapies), bioproduction, and vaccine adjuvants.

The invention provides methods and compositions deploying ddhNTPs for targeting natural and synthetic nucleotide-sensing pathways

In an aspects and embodiments the invention provides:
1. Design and use of ddhNTPs as immunomodulatory therapeutics to modulate (activate or inhibit) P2 receptors.
2. Design and use of ddhNTPs as nucleotidase inhibitors
3. Design and use of ddhNTPs for host-acting anti-infectives, oncolytics, anti-aging, tissue regeneration.
4. Design and use of CTP derivatives as immunomodulatory therapeutics to either activate or inhibit P2 receptors.
5. Design and use of CTP derivatives as nucleotidase inhibitors.
6. Design and use of CTP derivatives as host-acting anti-infectives, oncolytics, anti-aging, tissue regeneration.
7. Design and use of rigid sugar pucker chemistry as therapeutic immunomodulatory therapeutics to either activate or inhibit P2 receptors.
8. Design and use of rigid sugar pucker chemistry for targeted nucleotidase inhibitors.
9. Use of rigid sugar pucker chemistry for host-acting anti-infectives, oncolytics, anti-aging, tissue regeneration, immunomodulatory therapeutics, including adjuvants, etc.
10. Use of ddh ribose-containing molecules as immunomodulatory therapeutics to either activate or inhibit P2 receptors.
11. Use of ddh ribose-containing molecules as targeted nucleotidase inhibitors.
12. Use of ddh ribose-containing molecules for host-acting anti-infectives, oncolytics, anti-aging, tissue regeneration.
13. Design and use of engineered (synthetic) Viperins capable of producing a non-naturally occurring ddhATP.
14. Use of ddhATP as a more potent therapeutic for anti-infectives, oncolytics, anti-aging, tissue regeneration.
15. Use of rigid sugar pucker and purine (guanine or adenine) resistant to nucleotidase-mediated degradation.
16. Design and use of a microbial host to produce ddhATP.
17. Design and use of engineered bio-orthogonal ddhNTP derivatives and cognate P2 receptors as a control system in a eukaryotic cell.
18. Design and use of engineered phosphorylases to phosphorylate bio-orthogonal ddhN nucleosides in situ

The present invention provides the following:
1. A method comprising use of ddhNTPs as immunomodulatory therapeutics to modulate (activate or inhibit) P2 receptors.
2. A method comprising use of ddhNTPs as nucleotidase inhibitors.
3. A method comprising use of ddhNTPs for host-acting anti-infectives, oncolytics, anti-aging agents, or tissue regeneration agents.
4. A method comprising use of CTP derivatives as immunomodulatory therapeutics to either activate or inhibit P2 receptors.
5. A method comprising use of CTP derivatives as nucleotidase inhibitors.
6. A method comprising use of CTP derivatives as host-acting anti-infectives, oncolytics, anti-aging agents, or tissue regeneration agents.
7. A method comprising use of rigid sugar pucker chemistry as therapeutic immunomodulatory therapeutics to either activate or inhibit P2 receptors.
8. A method comprising use of rigid sugar pucker chemistry for targeted nucleotidase inhibitors.
9. A method comprising use of rigid sugar pucker chemistry for host-acting anti-infectives, oncolytics, anti-aging agents, tissue regeneration agents, or immunomodulatory therapeutics, including adjuvants.
10. A method comprising use of ddh ribose-containing molecule as an immunomodulatory therapeutic to either activate or inhibit P2 receptors.
11. A method comprising use of ddh ribose-containing molecule as targeted nucleotidase inhibitors.
12. A method comprising use of ddh ribose-containing molecule for host-acting anti-infectives, oncolytics, anti-aging, tissue regeneration.
13. A method comprising use of engineered (synthetic) Viperins to produce a non-naturally occurring ddhATP.
14. A method comprising use of ddhATP as a more potent therapeutic for anti-infectives, oncolytics, anti-aging agents, or tissue regeneration agents.
15. A method comprising use of rigid sugar pucker and purine (guanine or adenine) based ddhNTP to resistant nucleotidase-mediated degradation.
16. A method comprising use of a microbial host to produce ddhATP.
17. A method comprising use of engineered bio-orthogonal ddhNTP derivatives and cognate P2 receptors as a control system in a eukaryotic cell.
18. A method comprising use of engineered phosphorylases to phosphorylate bio-orthogonal ddhN nucleosides in situ.
19. A method of SynVip design strategy comprising template screening, in silico active site optimization and library curation for wet bench testing, and comprising steps:
a) construct in silico Viperin using AlphaFold;
b) perform HT docking to predict preferred substrate;
d) restrict to purine (GTP) accepting Viperins;
e) perform secondary screen to identify adenosine permissive Viperins;
f) identify all active site residues predicted to be within 6A of the docked Adenosine;
g) perform combinatorial screen to identify low energy binding conformations that increase interactions with ATP; and
h) curate final library of 20-30 SynVips maximizing structural diversity for wet bench testing.

The invention encompasses all combinations of the particular embodiments recited herein, as if each combination had been laboriously recited.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows the inhibition of expression via RNAP by ddh nucleotides via various Vip.

FIG. 2 shows [ATP] versus various ENTPD.

FIG. 3 shows [ddhC] versus various ENTPD.

FIG. 4 shows Pi release rate versus various NTP, 3′-dCTP, and ddhCTP.

FIG. 5 shows Pi release rate versus various NTP, 3′-dCTP, and ddhCTP.

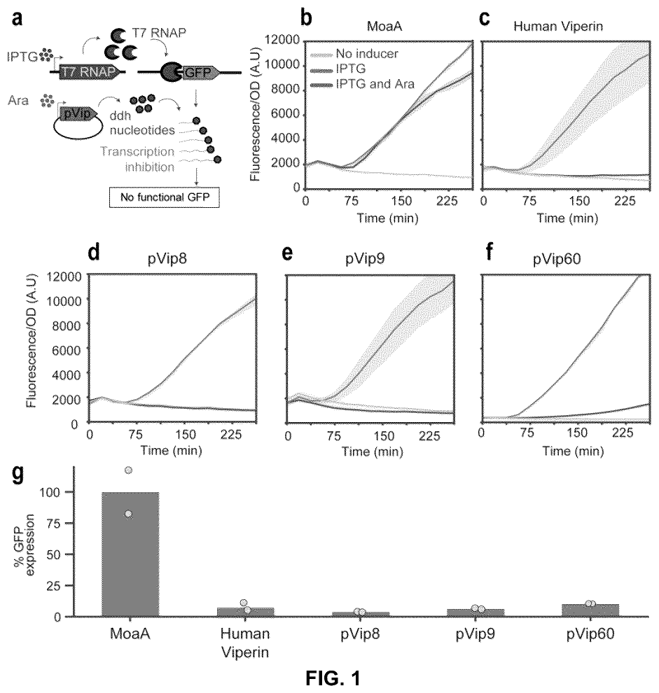

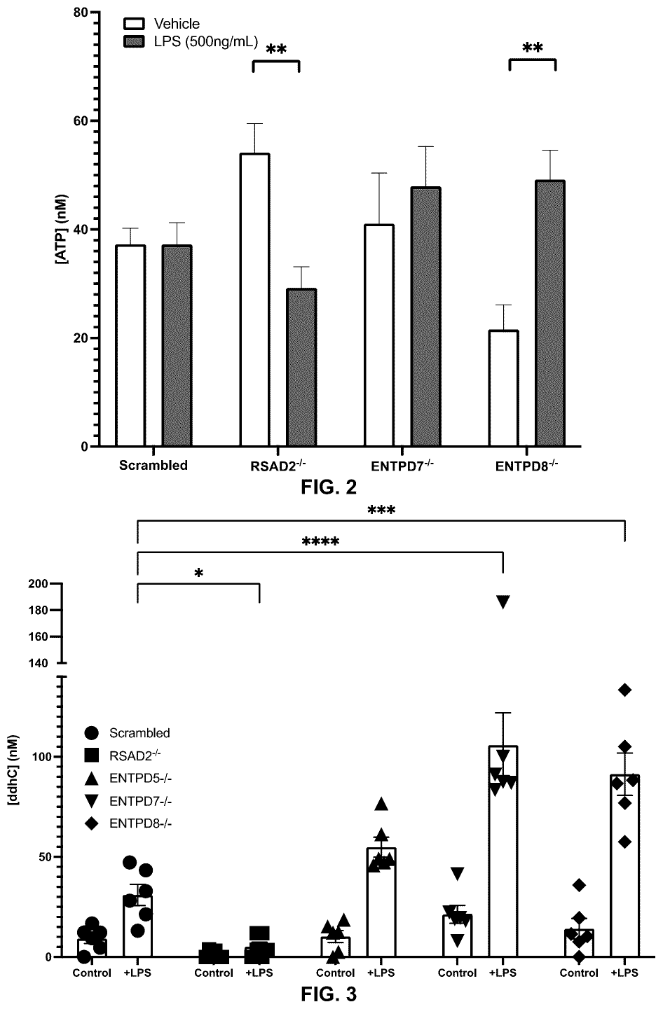

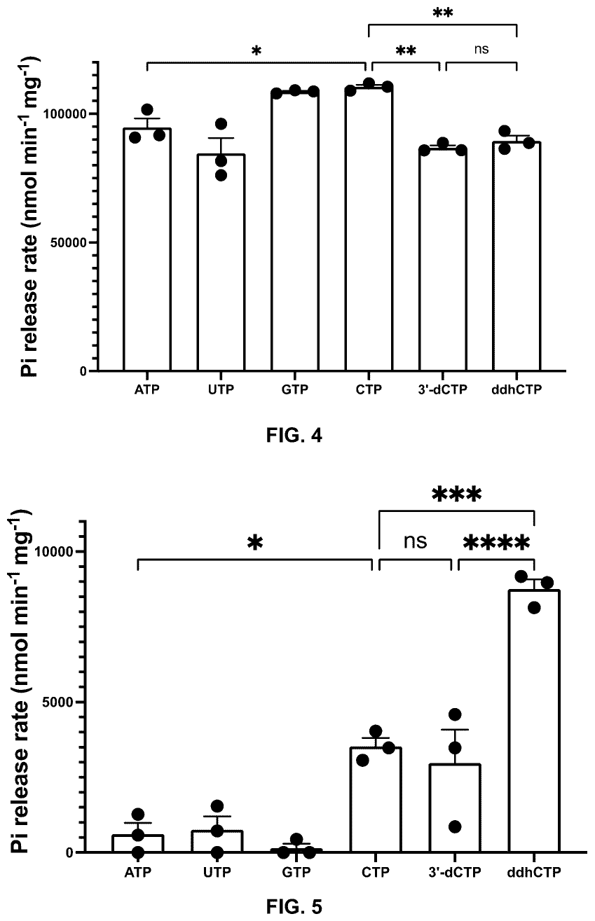

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Unless contraindicated or noted otherwise, in these descriptions and throughout this specification, the terms “a” and “an” mean one or more, the term “or” means and/or.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The terms “cell”, “host cell” and “host microorganism” are used interchangeably herein to refer to a living biological cell that can be transformed via insertion of an expression vector.

The term “heterologous” as used herein refers to a material, or nucleotide or amino acid sequence, that is found in or is linked to another material, or nucleotide or amino acid sequence, wherein the materials, or nucleotide or amino acid sequences, are foreign to each other (i.e., not found or linked together in nature).

The terms “expression vector” or “vector” refer to a compound and/or composition that transduces, transforms, or infects a host microorganism, thereby causing the cell to express nucleic acids and/or proteins other than those native to the cell, or in a manner not native to the cell. An “expression vector” contains a sequence of nucleic acids (ordinarily RNA or DNA) to be expressed by the host microorganism. Optionally, the expression vector also comprises materials to aid in achieving entry of the nucleic acid into the host microorganism, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host microorganism and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular species, but also encompasses a promoter from a corresponding gene in other species.

A polynucleotide is “heterologous” to an organism or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “operatively linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

In this disclosure, we demonstrate ddhCTP and its cognate nucleoside are ubiquitous in biological systems and contribute significantly to immune cell physiology by activating P2 receptors in a context-dependent manner. We disclose that the unique sugar pucker stereochemistry is a critical contributor to ddhCTP-mediated P2 receptor agonism and the targeted degradation of ddhCTP by ENTPD8, allowing host factors to discriminate this compound from other endogenous nucleotide pools. We also disclose this previously unrecognized signaling axis informs for the therapeutic activity of NHC (i.e. Molnupiravir), a cytidine nucleotide derivative shown to be effective in suppressing intracellular SARS-COV-2 replication in vitro. Our data indicates that Molnupiravir's therapeutic activity is partially mediated through P2 receptor agonism, as well.

The invention provides a novel approach for designing small molecules that either promote or inhibit P2 receptor-mediated effects on host physiology, including blood clotting, tissue regeneration, inflammation, vasodilation, neurological function, mucus secretion, angiogenesis, anti-pathogen protection, host resilience, and tumor suppression. Additionally, the invention provides novel design principles for developing inhibitors for nucleotidases that specifically target small molecules with similar chemical properties to that of ddhCTP and degradation-resistant purine-based ddhNTP compounds.

The invention provides other ddhNs that exhibit enhanced or augmented activity relevant to the naturally occurring ddhNTPs, including ddhCTP, ddhGTP, and ddhUTP. We disclose examples wherein ddhA is a more potent antiviral therapeutic relative to either ddhC or ddhG, for multiple RNA viruses. Given ddhATP production has not been found to occur by any naturally occurring enzymes, we engineered the Tenacibaculum todarodis Viperin homolog to accept ATP as a substrate in order to produce the non-naturally occurring ddhN nucleoside ddhA. We also demonstrate that a wide diversity of Viperin homologs can be expressed in the industrial host S. cerevisiae as a proof-of-concept for microbially-produced ddhNTP derivatives.

We performed extensive research and development of the various embodiments of the invention, empirically demonstrating inter alia:
ddhC treatment can be used to suppress intracellular mycobacterial pathogen growth; for example, we demonstrated that ddhC treatment reduces M. marinum abundance compared with cytidine 4 and 5 days post infection;
LPS can be used to stimulate release of non-ATP nucleotide pools; for example, we showed that ddhCTP is released into the extracellular space following LPS stimulation; RSAD2^{-/-} macrophages exhibit altered cytokine secretion profile and relA (NFkB) activity; NTPs can be used elicit different acute Ca²⁺ flux signatures in mouse sensory neurons; NTPs can be used to elicit unique Ca²⁺ flux signatures in conditionally-immortalized macrophages (CIMs); ddhCTP can be used as a potent ligand for P2Y and P2X receptors; basal ddhCTP-mediated NFAT signaling drives autophagy and pro-regenerative pathways; ddhCTP calcium flux is allosterically regulated and is mediated by P2XRs; ATP allosterically regulates P2X2 to permit CTP binding; ddhC antiviral activity is mediated by P2XRs; P2YRs enhance ZIKV production under physiologically relevant conditions; P2XRs do not substantially impact viral titers under physiologically-relevant conditions; P2XRs are sufficient to elicit ddhC-mediated protection; P2Ys are sufficient to elicit ddhC-mediated protection; and P2Rs are necessary for ddhC-mediated antiviral activity.

We also disclose, inter alia:
1. For nucleotidases (DNS): pyrimidine nucleotidases (ENTPD7 and ENTPD8) reduce basal NFkB activity; 3′ hydroxyl contributes to ENTPD7 substrate specificity; sugar pucker contributes to ENTPD8 substrate recognition; ENTPD8−/− CIMS exhibit enhanced autophagy flux; and nucleotidase knockout macrophages are more resistant to viral infection;
2. For P2 Receptors: CTP and ddhCTP elicit distinct calcium flux responses; ddhCTP-mediated calcium flux acts through Store Operated Calcium Entry (SOCE); ddhCTP stimulation elicits sustained Ca2+ flux activity on CIMs; and high concentrations of ddhC exhibit antiviral properties;
3. For ddhCTP: ddhC amplifies immune response in a P2X4-dependent manner; ddhC-mediated suppression of ZIKV replication is P2X4 dependent; ddhC-mediated suppression of ZIKV replication is not P2Y dependent (DNS); and ddhCTP-mediated calcium flux is driven by intracellular calcium stores (DNS).
4. For NHC: NHC amplifies immune response in a P2X-dependent manner; NHC-mediated effect is ˜200 fold more potent than ddhC; and NHC-mediated protection is more robust but less specific.

We also demonstrate: P2X4 ligand preference and NHC structure-activity relationship; rational design of unnatural ddhNTP derivatives by, for example, functionalizing N4 (ddhCTP) or N6 (ddhATP) with electron withdrawing groups (EWGs) to generate more potent, more specific P2X4 agonist than NHC.

The invention also provides a SynVip design strategy: template screening, in silico active site optimization and library curation for wet bench testing:
1. Construct In silico Viperin using AlphaFold2;
2. Perform HT docking to predict preferred substrate;
3. Restrict to purine (GTP) accepting Viperins;
4. Perform secondary screen to identify adenosine permissive Viperins;
5. Identify all active site residues predicted to be within 6A of the docked Adenosine;
6. Perform combinatorial screen to identify low energy binding conformations that increase interactions with ATP; and
7. Curate final library of 20-30 SynVips maximizing structural diversity for wet bench testing.

The invention also provides, inter alia:
1. Use robotics facility to perform automated build for ddhATP production hosts;
2. Wet bench testing of final SynVip library (LC-MS/MS); and/or
3. Construction of ddhATP reporter line for SynVip enzyme optimization.

derivatize and test ddhA compound family

We also disclose, inter alia:

RSAD2 and ENTPD8 contribute to extracellular ATP levels; Pyrimidine nucleotidases are expressed on macrophages;

ENTPD7 and ENTPD8 significantly increase extracellular ddhC nucleoside residence time;

Nucleotide structure-activity relationship: 3′ hydroxyl contributes to ENTPD7 substrate specificity; Sugar pucker contributes to ENTPD8 substrate specificity; and

Nucleotidase knockout macrophages are more resistant to ZIKV infection.

In additional experimental results we demonstrated and confirmed: ddhCTP is a natural ligand of purinergic receptors; P2 receptors modulate host immunity and pathogen clearance in a context-dependent manner; ENTPD8 preferentially metabolizes ddhCTP and influences viral replication; and ENTPD8 contributes to colorectal cancer tumorigenicity.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### ddhCTP Stereochemistry Influences Cell Physiology and Metabolism in Diverse Eukaryotic Tissues

Here, we demonstrate ddhCTP and its cognate nucleoside are ubiquitous in biological systems and contribute significantly to mammalian cell physiology by activating nucleotide-sensing purinergic (P2) receptors. We also demonstrate that Entpd8, an extracellular nucleotidase recently reported to counteract P2R-mediated tissue inflammation (Tani et al., 2021), preferentially degrades ddhCTP that is dependent on the rigid ribose pucker characteristic of the ddhNTP chemical family.

ddhCTP is a natural P2R ligand and contributes to ddhC-mediated antiviral activity

Ribonucleotides are important extracellular signaling molecules that modulate cell physiology via intracellular calcium flux, and are sensed by a wide array of extracellular purinergic (P2R) receptors. However, the ligands historically reported to activate P2 Receptors are limited to either ATP or UTP. To determine whether ddhCTP is a natural ligand of P2 receptors, primary murine neurons were stimulated with either CTP or ddhCTP and intracellular calcium concentration was measured over time. Surprisingly, ddhCTP simulated a calcium oscillatory pattern distinct from that observed with CTP stimulation, indicating extracellular sensory factors respond to ddhCTP differently that CTP, ddhCTP's metabolic precursor.

Calcium flux into the cytosol mediates a wide range of physiological responses, including cell motility, muscle contraction, neuronal transmission, cell growth, and inflammatory responses. Cytosolic calcium influx can originate from either the extracellular environment or intracellular organelle calcium stores, depending on which host factor is responsible for sensing a given stimulus. P2 Receptors are subdivided into two distinct families, P2Y Receptors, a class of GPCRs that upon activation release intracellular calcium stores into the cytosol, and P2X Receptors, ligand-gated cation channels that mobilize calcium from either the extracellular or intracellular environment. To determine the source of calcium influx, primary neurons were cultured in either 2 mM Ca²⁺ or Ca²⁺ depleted buffer chelated with EGTA. Primary neurons were then stimulated with CTP, ddhCTP, or 3′-dCTP, a CTP derivative that relaxes the constrained ddhCTP ribose ring, and the proportion of primary neurons responding to stimulation was recorded. Neurons stimulated with CTP only responded when extracellular Ca²⁺ was present, indicating that CTP-mediated calcium flux was solely dependent on ligand-gated plasma calcium channels localized on the plasma membrane. Neurons stimulated with 3′-dCTP revealed a similar response in calcium replete buffer, but also showed a small subset of cells that responded in Ca²⁺ depleted buffer, indicating that some neurons were able to mobilize calcium via intracellular calcium stores. Compellingly, neurons stimulated with ddhCTP revealed a small proportion of responding cells that was independent of extracellular calcium, indicating ddhCTP-mediated calcium flux is dependent on intracellular calcium stores. Thus, these data indicated extracellular CTP and ddhCTP are sensed by neurons via P2X and P2Y receptors, respectively.

To determine which P2Y receptors specifically respond to ddhCTP, a library of P2Y-expressing 1321N1 cell lines were individually stimulated with 1 uM ddhCTP. Calcium flux responses were observed for those cell lines expressing P2RY1, P2RY2, and P2RY4, but not P2RY6, P2RY11, and P2RY12. These data demonstrate that ddhCTP is a natural extracellular ligand for P2Y receptors.

ddhCTP was initially reported to be an endogenous chain terminator that disrupted flavivirus RdRPs. To evaluate the contribution of P2R signaling to ddhCTP's previously observed antiviral activity, VeroE6 cells were infected with ZIKV either with or without 1 mM ddhC supplementation and with or without P2R inhibitors. As previously reported, ddhC supplementation significantly suppressed ZIKV replication by 72.2±6.5%. Surprisingly, when ddhC-treated cultures were infected in the presence of P2RY2 and P2RX4 inhibitors, no significant difference in viral titers were observed when compared to cultures treated with P2R inhibitors only. Conversely, ddhC-mediated therapeutic activity was still observed in the presence of P2RY6 inhibitor.

To orthogonally evaluate whether P2Rs contribute to ddhC's antiviral activity, P2R-expressing 1321N1 cell lines were pretreated with 1 mM ddhC or vehicle control for 24 hours and were subsequently infected with ZIKV. The parent 1321N1 that naturally lacks P2R expression was also included as a negative control. After 48 hrs, supernatant was collected, and live viral titer was determined via cytopathic effect. ddhC treatment did not significantly reduce viral replication for the parent 1321N1 line (13.2±22.7%). However, ddhC treatment significantly reduced viral replication by 79.8±10.8%, 86.9±3.1%, 87.3±2.9%, and 37.3±15.6% for 1321N1 lines heterologously overexpressing P2RY1, P2RY2, P2RY4, and P2RX4, respectively. Additionally, co-expression of P2RX2, a nucleotide receptor known to form heteromers with P2RX4, with P2RX4 further reduced viral replication by 68.9±6.0%. Taken together, these data indicate ddhC is a natural ligand for nucleotide receptors and these receptors synergistically interact with ddhC to suppress viral replication in mammalian cells.

P2 Receptors modulate host immunity and pathogen clearance in a context-dependent manner

The data above indicate high concentrations of ddhC can elicit P2R-dependent antiviral activity. However, recent studies report that P2ry2 and P2rx5 have disparate influences on HCMV replication: P2ry2 increases viral replication by an order of magnitude while P2rx5 inhibits viral replication by roughly the same magnitude (Chen et al., 2019). This study also identified multiple mechanisms through which P2ry2 contributes to viral replication, suggesting one or some of these mechanisms could be exploited by other viral pathogens. Indeed, we also found that treating Vero-E6 cells with the P2ry2 inhibitor AR-C 118925XX throughout infection significantly reduced viral load by approximately 50.9%. Moreover, recent studies quantifying ddhC in human blood serum report ranges of 45 nM in healthy donors to 970 nM in COVID-19 patients (Roberts et al., 2023) suggest the dosing regimen used in our initial experiments may be superphysiological in nature.

To determine whether ddhC contaminants are present in standard medium supplements, we measured ddhC levels in culture medium supplemented with 10% heat inactivated fetal bovine serum and detected concentrations of 31.2±4.0 nM, indicating the levels of ddhC present in standard culture medium is more physiologically relevant than those used in our initial experiments. Given these discrepancies, we sought to investigate the way by which P2 receptors contribute to host immunity and pathogen clearance under physiologically relevant conditions.

To that end, P2R-expressing 1321N1s were infected with ZIKV (MOI=0.1). After 48 hrs, supernatant was collected, and live viral titer was determined via cytopathic effect (CPE). Surprisingly, the P2RY1-, P2RY2-, and P2RY4-1321N1 lines revealed a 494±85%, 351±90%, and 205±78% increase in viral replication relative to the 1321N1 parent line (100±65%). The P2RY6-1321N1 line also showed a slight increase in viral titer (177±55%), but this trend was not statistically significant across multiple experiments. Similarly, supernatant viral titers after 48 hrs of the P2RX2-, P2RX4-, and P2RX2/P2RX4-1321N1s were 80±65%, 101±35%, and 110±52% relative to that of the parent line.

Although consistent, the experiments above relied upon cell lines and artificial methods of modulating nucleotide receptor activity during infection. Thus, we sought to see whether these findings were consistent in primary cells, since these models more faithfully recapitulate cell signaling processes in a natural setting. In working toward that end, we decided to employ macrophages, a well-established model for studying innate immunity and host-pathogen interactions. Bone marrow derived macrophages (BMDMs) were stimulated with either PAM, a TLR2 agonist mimicking bacterial infection, or Poly (I: C), a TLR3 agonist that mimics RNA virus infection, for 24 hours. Bulk RNA-seq was then performed to identify relevant P2 receptors. Our data revealed that P2ry2, P2ry4, P2ry6, P2ry14, P2rx2, P2rx4, and P2rx6 were all expressed in BMDMs, and numerous receptors were differentially regulated in a TLR ligand-dependent manner.

It is well-established that infection or PAMP stimulation of primary macrophages cause an increase in extracellular ATP concentrations that activate P2 receptor-mediated inflammation. To determine whether Viperin contributes to increased levels of extracellular ddhCTP concentrations in situ, Rsad2^{-/-} macrophages were established using conditionally-immortalized macrophages (CIM), a transgenic cell line which contains both an estrogen-inducible HoxB8 transgenic element for continuous propagation as well as a constitutive Cas9 genetic element for efficiently generating genetic knockouts. Our previous work demonstrated CIM physiology is remarkably similar to that of bone marrow-derived macrophages (BMDMs), a commonly used model for studying innate immunity and host-pathogen interactions (Roberts et al., 2018). Rsad2^{-/-} CIMs and control CIMs transduced with scrambled guides were then stimulated with 50 ng/ml LPS. Supernatants were then collected after 24 hours and ddhCTP levels were measured via LC-MS/MS. Detectable levels of ddhCTP were not observed, likely due to the endogenous nucleotidase activity inherent in the system and instrument sensitivity required to detect nanomolar concentrations; however, ddhC was detected in the supernatant, and elevated levels of extracellular ddhC were observed in supernatants harvested from LPS-stimulated scramble guide-transduced macrophages relative to unstimulated control (63.8±12.7 pmol per 10⁶ cells vs. 18.4±6.0 pmol per 10⁶ cells, respectively). Conversely, elevated levels of ddhC were not detected in either unstimulated or LPS-stimulated Rsd2^{-/-} macrophages (4.6±2.8 pmol per 10⁶ cells vs. 15.9±6.3 pmol per 10⁶ cells), indicating endogenous Rsad2 expression has the capacity to modulate extracellular ddhC levels in situ.

Previous studies report Rsad2^{-/-} BMDMs have dysregulated cytokine production profiles in response to TLR stimulation (Eom et al., 2018). To determine whether loss of Viperin caused immunodysregulation in our model, Rsad2^{-/-} and scrambled guide control macrophages were stimulated with 50 ng/mL LPS and supernatants were collected after 24 hours. TNFa, CXCL10, and IL-1B secretion were then detected via ELISA. In agreement with previous studies, TNFA secretion was significantly increased for Rsad2/CIMs relative to scrambled controls (2193+47 μg/mL vs. 966+28 μg/mL), while CXCL10 secretion was significantly reduced relative to scrambled controls (340+36 μg/mL vs. 15057+1940 μg/mL).

Having identified those nucleotide receptors expressed in macrophages, P2R knockout lines were established in CIMs. Each line was then infected with ZIKV (MOI=1) for 48 hours, after which the supernatant was collected, and live viral titers were determined via cytopathic effect. In agreement with previous data, P2ry2^{-/-} and P2ry4^{-/-} macrophages produced significantly lower viral titers (62.4±6.4% and 52.7+10.9%, respectively) than scrambled controls. P2ry6^{-/-} macrophages exhibited a 35.3±9.9% reduction in viral titer, but this trend failed to reach statistical significance across experiments. In agreement with experiments performed with 1321N1s and VeroE6s, P2rx2^{-/-} and P2rx4^{-/-} macrophages also failed to show differences in viral titer under physiologically-relevant concentrations of ddhC. Finally, Rsad2^{-/-} macrophages revealed a 37.9±16.4% increase in viral titers relative to controls; however, this trend failed to reach statistical significance across experiments, possibly due to contaminating ddhC in the medium that partially obscures ddhC-mediated phenotypic changes in vitro.

From the experiments above, we identified P2ry2 and P2rx4 as two concentration-dependent contributors to viral replication, with P2ry2 promoting viral replication at lower ligand concentrations, and P2rx4 inhibiting viral replication at higher concentrations. However, previous reports have shown P2Y and P2X receptors have both redundant and divergent influences on cell physiology.

One compelling similarity is that both P2ry2 and P2rx4 receptors have previously been found to contribute to cellular dysfunction and disease pathology by inhibiting autophagy (Dusabimana et al. 2020; Zhang et al., 2021). To determine whether P2Rs may contribute to autophagy-mediated pathogen clearance, P2R-null CIMs were infected with a mCherry-tagged Triple Mutant Listeria monogenes strain lacking the pore-forming cytolysin listeriolysin O (LLO) gene, the phosphatidylinositol-specific phospholipase C (PlcA) gene and the promiscuous, phospholipase C (PlcB) gene. This model was previously used to study autophagic activation and autophagy-mediated bacterial clearance in primary macrophages (Mitchell et al., 2018).

Colocalization of LC3 and mCherry was quantified as a proxy for autophagic targeting of triple mutant L. monogenes in Rsad2^{-/-}, P2ry2^{-/-} and P2rx4^{-/-} CIMs. Compared to controls, only P2rx4-deficient CIMs revealed a significant increase in colocalization, indicating P2rx4 is the predominant inhibitor of autophagy-mediated pathogen clearance in macrophages. Interestingly, cellular pathogen burden as measured by mCherry showed a significant decrease in all knockout backgrounds, indicating autophagy-independent mechanisms of pathogen clearance are inhibited by Rsad2^{-/-} and P2ry2^{-/-} .

NF-kB signaling has been shown to be an important contributor to L. monogenes clearance (Sha et al., 1995). Considering the primed inflammatory state observed in Rsad2^{-/-} and P2ry2^{-/-} macrophages at the transcriptional level, we sought to determine whether basal NF-kB activity may be elevated in Rsad2^{-/-} and P2ry2^{-/-} macrophages by establishing a synthetic reporter composed of a minimal promoter and tandem relA binding domains in CIMs. Rsad2^{-/-} and P2ry2^{-/-} CIMs were then generated this relA reporter line along with matching scrambled controls. In agreement with our hypothesis, an elevated level of relA activity was observed in Rsad2^{-/-} and P2ry2^{-/-} cells relative to scrambled controls. Flow cytometric analysis of Rsad2^{-/-} cells revealed a 60.3±10.6% increase in basal relA activity relative to control. LPS stimulation increased reporter activity by 53.4±7.1% and 93.2±19.3% relative to unstimulated controls, for scrambled and Rsad2^{-/-} cells, respectively.

Taken together, these data indicate P2Y and P2X receptors are important dose-dependent contributors to modulating macrophage physiology through disparate mechanisms: P2ry2 actively suppresses central mediators of inflammation, such as p65 activity, while P2rx4 directly inhibits autophagy-mediated pathogen clearance, both of which have been reported in recent literature (Nishida et al., 2011; Zhang et al., 2021; Kawano et al., 2012).

ENTPD8 Preferentially Metabolizes ddhCTP and Influences Viral Replication

Membrane-bound nucleotidases act to resolve receptor-mediated inflammation by hydrolyzing purinergic nucleotides in the extracellular environment. To determine which of these enzymes may contribute to resolving purinergic inflammation in macrophages, the BMDM transcriptome was surveyed, and detectable levels of Entpd1, Entpd5, Entpd7, and Entpd8 were observed. Compellingly, the most highly expressed nucleotidase was Entpd8, a poorly studied nucleotidase that is highly expressed in myeloid and intestinal tissue.

To first evaluate whether the two most highly expressed nucleotidases increase residence time of ddhC in the extracellular environment, Entpd7^{-/-} and Entpd8^{-/-} CIM lines were established. These lines were then stimulated with 50 ng/mL LPS for 24 hours to intracellular release of intracellular nucleotide pools into the extracellular environment. LC-MS/MS analysis revealed supernatants harvested from LPS-stimulated Entpd7^{-/-} and Entpd8^{-/-} macrophages contained elevated ddhC levels relative to scrambled controls.

Nucleotidases are ubiquitous in biological systems and have distinct substrate binding preferences for both naturally occurring and synthetic nucleotide derivatives. Previous reports have found that some nucleotidases prefer nucleotides with specific ribose ring conformations, or “ribose puckers” (Salmaso and Jacobson, 2020). Of particular significance, it has been reported that some of these proteins prefer synthetic nucleotides with locked ribose pucker conformation, although there has been no natural explanation for why these proteins evolved to prefer these substrate preferences (Maruoka et al., 2010; Jacobson et al., 2001). To evaluate whether the unique ribose pucker of ddhCTP may have relevance to Entpd7 and Entpd8, the differential degradation of ddhCTP relative to other nucleotides was determined in vitro. 3′-dCTP, a ddhCTP derivative that saturates the 4′-3′ double bond and relaxes the ddhCTP sugar pucker, was included as a control to determine whether the ddhCTP sugar pucker contributes to its recognition as a substrate. Entpd7-mediated degradation of ddhCTP was observed to be 89.1±5.3% compared to that of ddhCTP and was sugar pucker independent. Degradation rates of the other nucleotides were also similar, indicating Entpd7 is a promiscuous enzyme that degrades extracellular nucleotides similarly. Compellingly, reactions with purified Entpd8 revealed that degradation of ddhCTP was 184.9±41.7% relative to that of CTP and this substrate preference was sugar pucker dependent. Degradation rates of the other nucleotides was similar to that of CTP, indicating Entpd8 is functionally linked to ddhCTP in mammalian systems.

To better understand the physiological role of ddhCTP in the context of infection, Entpd7^{-/-}, Entpd8^{-/-}, and Entpd7^{-/-}; Entpd8^{-/-} were infected with ZIKV (MOI=1) for 48 hours, after which the supernatant was collected and live viral titer was determined via cytopathic effect. Live viral titers were found to be 47.2±6.7%, 51.6±5.5%, and 41.8±7.0% of scrambled controls, respectively.

### Entpd8 Promotes Cancer Cell Resilience

Entpd8 has recently been identified as a reliable prognostic biomarker in colorectal cancer patients (Yi el al., 2022), and high expression is associated with poor outcomes as measured by five year survival. However, it is unclear whether Entpd8 directly contributes to colorectal cancer pathogenesis or is downstream of those factors that drive disease progression. RKO cells are an established colorectal cancer cell model previously used to examine the tumorigenic role of CD73 (Wu et al., 2015), another extracellular nucleotidase, and reportedly lacks endogenous expression of ENTPD8, indicating this is an appropriate model to test the contribution of ENTPD8 to colorectal cancer cell physiology. To that end, lentiviral expression cassettes were used to establish stable cell lines that constitutively overexpress wildtype ENTPD8 and variants that exchange residues thought to form hydrogen bonds with ddhCTP with other residues from other known nucleotidases that exhibit alternative substrate affinities.

Colony formation assays were performed by plating 500 cells per well and incubating cultures for 10 days. Colony formation efficiency was quantified as previously described (Wu et al., 2015). Cell lines overexpressing ENTPD8 were observed to have a greater number of colonies per well relative to the pLenti backbone transduced control. This increase in clonogenicity was lost in cell lines expressing a mutant that disrupted one or two hydrogen bonding residues. Compellingly, one triple mutant that swapped three interacting R-groups in ENTPD8 for those present in ENTPD1 exhibited similar clonogenicity to that of ENTPD8, indicating swapping of all three interacting domains increased nucleotide binding affinity by making the active site more promiscuous.

### Example 2

### Industrial Application of Nucleotide Derivatives Inspired by the Non-Canonical Nucleotide ddhCTP

The host-pathogen arms race has laid the foundation for fundamental mechanisms by which higher organisms protect themselves from infection and recover from injury. Among the most ancient modulators of innate immunity is Viperin, an interferon-stimulated gene (ISG) historically reported to elicit broad-spectrum antimicrobial activity. Recent studies investigating the role of Viperin in mammalian systems have identified it as one of the few Radical SAM enzymes encoded by the human genome. Viperin confers host protection by catalyzing an energetically unfavorable free radical reaction that produces 3′-Deoxy-3′,4′-didehydro-cytidine triphosphate (ddhCTP), a novel, non-canonical nucleotide with unique stereochemistry arising from a 4′-3′ double bond on the ribose ring. Bioprospecting efforts later revealed that Viperin homologs are represented across all domains of life, and these protein variants produce ddhNTPs containing alternative nucleobases. These findings suggest that this unique chemical family holds broader significance than initially described. Early reports on ddhCTP's mechanism of action proposed that this nucleotide suppresses viral replication by interfering with RNA-dependent RNA polymerases. However, given the ubiquity of these molecules in nature, I sought to explore whether ddhCTP also exerts host-directed effects, and if so, whether it influences the physiology of other higher eukaryotes, such as plants. My initial investigations revealed that ddhCTP acts as a natural ligand for Type 2 purinergic receptors (P2Rs), and that one extracellular nucleotidase, Entpd8, uniquely recognizes ddhCTP due to its distinctive “sugar pucker” stereochemistry. Furthermore, I show that exposing A. thaliana to ddhC throughout its growth cycle results in longer roots and increased leaf number at bolting—two indicators of improved crop health. Building on these findings, I designed novel technologies aimed at enhancing crop resilience, improving drug efficacy, and developing living therapies. For instance, I leveraged the specific interaction between ddhCTP and Entpd8 to develop a potent Entpd8 inhibitor that suppresses cancer cell survival. I then demonstrate that the sugar pucker of ddhNTPs enhances antiviral activity and optimize the parent ddhCTP scaffold to further amplify these effects. Finally, I engineered Viperin fusion proteins that produce elevated titers of ddhCTP, and validated these constructs in the anaerobic gut commensal Bacteroides thetaiotaomicron, thereby establishing a living therapeutic capable of delivering ddhC systemically via the gastrointestinal tract. Collectively, these findings and resulting tools offer proof-of-concept that the ddhCTP chemical family holds broad utility across the pharmaceutical, agricultural, and biotech sectors.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method to modulate a P2 receptor comprising administering or introducing a ddhNTP or ddhN as an immunomodulatory therapeutic to a host to modulate a P2 receptor.

2. The method of claim 1, wherein to modulate is to activate.

3. The method of claim 1, wherein to modulate is to inhibit.

4. The method of claim 1, wherein a ddhNTP is introduced and the ddhNTP is ddhATP, ddhUTP, ddhGTP, or ddhCTP.

5. The method of claim 1, wherein the ddhNTP is ddhCTP.

6. The method of claim 1, wherein a ddhN is introduced and the ddhN is ddhA, ddhC, or ddhG.

7. The method of claim 1, wherein the ddhN is ddhA.

8. The method of claim 1, wherein the P2 receptor is P2RY1, P2RY2, P2RY4, or P2RX4.

9. The method of claim 1, wherein the modulation of the P2 receptor promotes or inhibit P2 receptor-mediated effects on host blood clotting, tissue regeneration, inflammation, vasodilation, neurological function, mucus secretion, angiogenesis, anti-pathogen protection, host resilience, and/or tumor suppression.

10. A method to inhibit a nucleotidase comprising contacting a ddhNTP to a nucleotidase whereby the nucleotidase is inhibited.

11. A method to modulate a P2 receptor comprising administering or introducing a CTP as an immunomodulatory therapeutic to a host to modulate a P2 receptor.
